(19) United States
(12) Patent Application Publication
(10) Pub. No.: US 2015/0297080 A1
(43) Pub. Date: Oct. 22, 2015

(54) NON-INVASIVE PORTABLE SYSTEM FOR THE MONITORING AND PRELIMINARY DIAGNOSIS OF ELECTROCARDIAC EVENTS IN REAL TIME

(71) Applicant: UNIVERSIDAD PONTIFICIA BOLIVARIANA, Medellín (CO)

(72) Inventors: Adrian Alberto Amaya Casas, Medellin (CO); John Jairo Bustamante Osorno, Medellin (CO); Sergio Albeiro Marin Correa, Medellin (CO); Jose Francisco Saenz Cogollo, Medellin (CO); Henry Hermel Andrade Caicedo, Medellin (CO)

(21) Appl. No.: 14/428,738

(22) PCT Filed: Sep. 18, 2013
(86) PCT No.: PCT/IB2013/058648

§ 371 Date: Jun. 2, 2015

(30) Foreign Application Priority Data
Sep. 20, 2012 (CO) 12163284

(51) Int. Cl. A61B 5/00; A61B 5/044; A61B 5/04; A61B 5/0404; A61B 5/11; A61B 5/046; A61B 5/0464; A61B 5/0432; A61B 5/0408

(52) U.S. Cl. CPC A61B 5/0006; A61B 5/04325; A61B 5/044; A61B 5/04017; A61B 5/0408; A61B 5/7203; A61B 5/0022; A61B 5/7228; A61B 5/1112; A61B 5/002; A61B 5/046; A61B 5/0464; A61B 5/0404; A61B 2560/0214

(57) ABSTRACT

The present invention discloses a portable system for acquiring, processing, storage, diagnostics, remote alarm transmission and cardiac event in patients, which operates at a sampling rate of a minimum 1 kHz signals, making it quick and effective. The system is non-invasive and capable of detecting more than eight cardiac conditions, unlike other similar devices on the market.

### 1. FIELD OF THE INVENTION

The present invention relates to a portable and non-invasive system for monitoring, storage, wireless remote communication and alarm of biometric data within a patient, and more particularly in electrocardiographic signals and transmission via a mobile data communication system, Bluetooth and GPS. The present invention also relates to a method for the monitoring, storage, remote communication and alarm of electrocardiographical events in patients, it is non-invasive and it directly transfers data via a mobile data communication system, Bluetooth and GPS reception.

### 2. DESCRIPTION OF THE STATE OF THE ART

The outcome of patients with cardiac arrhythmia disease requires constant medical monitoring which involves access techniques and monitoring methods, many of which involve the patient's stay in a hospital setting. Cardiac monitoring equipment used in these units are not portable and require the patient to be confined in an enclosure for long periods of time. One available solution is the Holter monitor that allows recording of cardiac activity of patients through an ambulatory way.

The Holter monitor permanently records both events in which the patient feels bad as well as when he or she feels normal and has the ability to manually activate a switch in the register of the cardiac signals in order to indicate abnormal symptoms. This feature differs as does the internal software (firmware) of the present invention and allows the cardiologist to focus solely on such events, either in real time or as a set of packs, given the unit also allows the storage of relevant information, unlike the Holter monitor which continuously records for a defined period of time (usually 24 or 48 hours) throughout the cardiac behavior, either normal or abnormal which the cardiologist then must review minute by minute to see if there were problems.

Another device of the prior art is the Braemar ER920 event monitor, considered one of the most advanced in the world, which further provides documentation of asymptomatic and symptomatic cardiac events, where the patient activates recording transient cardiac events. This Braemar device does not have the ability to transmit information in real time from any place. The signal information is stored in an internal memory to be later downloaded to a computer by a cable when possible. The difference between the present invention and the Braemar ER920 device lies in the communication platform, since the present invention has the ability to integrate detection of electrocardiographic signals with real-time communication via a cellular network.

There is also a device for detecting cardiac events called TZMedical, currently available in the market. The main difference with the present invention is that this equipment detects only 4 types of cardiac pathologies, while the present invention detects even up to 8 different types of pathologies. Furthermore, the TZMedical works at a frequency ranging between 256-512 Hz, while the equipment of the present invention operates at least at 1 kHz, making it quicker and providing more detailed readings. In another differentiating aspect, the TZMedical computer does not have a Bluetooth geo localization system connection and GPS while the present invention is capable of transmitting via Bluetooth to any nearby computer and also captures the patient's georeference and geolocation information every time the device detects a cardiac event.

Patent No. US2010/0145161 A1 describes a system for remote monitoring of patients using wireless networks. This invention focuses on capturing the vital signs of a patient but does not specialize in detecting some type of trauma, complications, failure or malfunction of the internal organs of said patient. It is specially worth noting that said device does not provide any method in diagnosing any type of heart disease. On the other hand, the system's implemented communication depends explicitly on the existence of a short-range network such as Wifi or non-cellular systems such as WiMax; which are very different from those used in this invention.

Patent No. WO2011/082341 A1 describes a similar system to the one mentioned above. Said patent describes a sensor network but does not indicate any system or specialized device in the diagnosis of heart disease through an electrocardiographic signal.

Patent No. US 2011/0125040 A1 describes a device that can perform monitoring of an electrocardiographic signal, but this device is not autonomous in view that its proper function depends on a conventional cellular computer on which a software is installed; clearly indicating that the device is not autonomous like the present invention is.

To further complement the above mentioned, patent number U.S. Pat. No. 7,212,849 B2 discloses a device for detecting arrhythmias. This device needs to be implemented by a surgical method which makes it different from the present invention given it does not require any specialized procedure in order for it to operate and function.

In light of the above techniques and prior art methods, these do not provide the advantages of the present invention. In fact, none of the previously shown mention the integration of web platforms and databases for the specialized medic to interact with the patient in case the device registers any anomalies, in addition to storing the historical record of the patient in a medical history.

### 3. BRIEF DESCRIPTION OF THE FIGURES

FIG. 1 shows the essential elements of a portable system for acquisition, processing, storage, diagnostics, remote alarm transmission and electrocardiographic events in patients of the present invention.

FIG. 2 shows a detailed version of the acquisition, processing and storage systems included in the device as functional components and further indicating which systems have an interrelation function.

FIG. 3 shows the function of each electronic element inside the device. Said figure indicates the components of each circuit element and their distribution.

FIG. 4 shows a flowchart of how the device is operated. Said flowchart indicates the steps to make the device work and further shows the tasks it develops while operational.

### 4. SUMMARY OF THE INVENTION

The present invention discloses a portable biomedical telemetry system for the acquisition, storage, remote communication, alarm processing and preliminary diagnosis of at least eight (8) electrocardiographic events in patients with heart problems, with the characteristic of being non-invasive and allows the downloading of the recorded data by a mobile communication system data, Bluetooth, and location through a global positioning system or GPS.

In one embodiment of the invention, a portable non-invasive device is provided in any part of the patient's body for the acquisition, processing, storage, and transmission of diagnostic electrocardiographic signals is described. The present invention further includes a wireless communication network which enables simultaneous data transmission via a mobile communication system such as Bluetooth and GPS location. The present invention also discloses the use of a data storage software hosted on a server, wherein the latter can be accessed via the web. The present invention also discloses an electrocardiographic signal visualization software characterized in that it allows the display of the information obtained from the patient at least in one mobile device in real time.

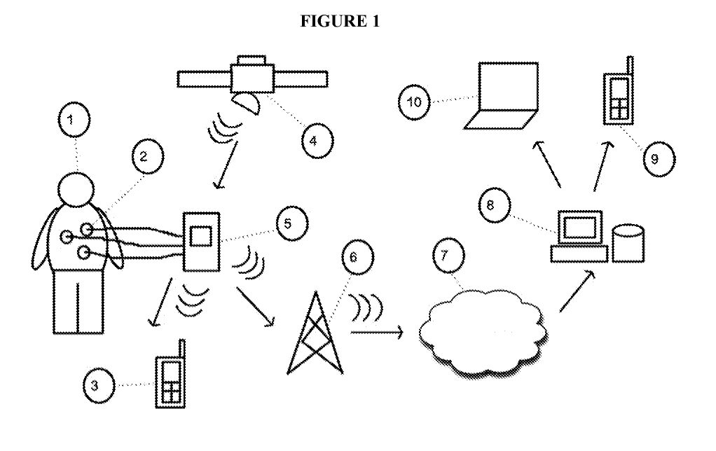

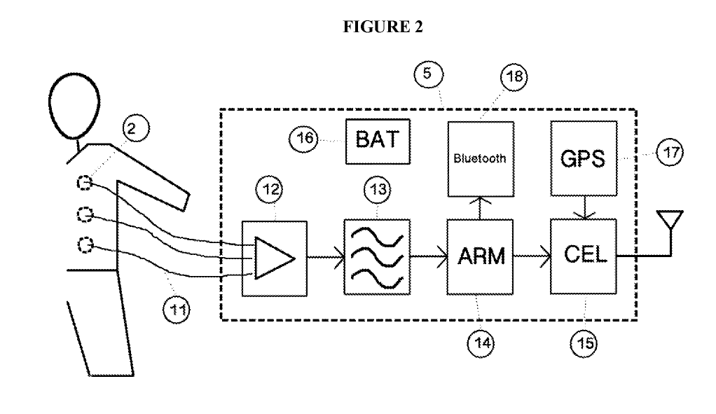

In another aspect of the present invention, and referring to FIGS. 1 and 2, the present invention's non-invasive portable device is characterized in that it comprises: i) a non-invasive signal acquisition system (2) of a patient's electrocardiographic events; ii) a processing and digital storage system of said electrocardiographic signals from said non-invasive portable device (5), and functionally linked to the non-invasive signal acquisition system (2) of electrocardiographic events; iii) a communications module (15) between the system of digital signal processing and preliminary diagnosis (14) and a wireless communication network (6) for the direct transmission of said electrocardiographic signals; and iv) an electrical power system (16) for the energy supply of the device.

In another aspect of the present invention, the non-invasive portable device of the present invention is further characterized by the non-invasive signal acquisition system (2) and diagnostic of electrocardiographic events further comprising: i) at least three electrodes for the acquisition of the electrocardiographic signals which are directly attached to a patient at different parts of the body; ii) an electrocardiographic signal amplifier (12) functionally connected to said electrodes; iii) a connecting means (11) between the electrodes and the electrocardiographic signals amplifier; and iv) at least one analog filter (13) adapting said electrocardiographic signals.

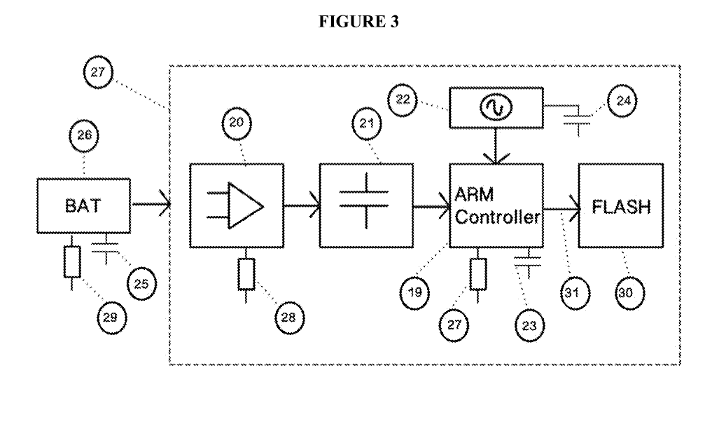

Continuing with FIGS. 2 and 3, the portable non-invasive device of the present invention is characterized in that the digital signal processing and preliminary diagnosis system (5) further comprises: i) a microcontroller for signal processing comprising at least one 32-bit ARM controller architecture to capture a signal of a minimal frequency of 1 kHz (14); ii) at least one signal amplifier functionally connected to said microcontroller (12); iii) at least one capacitor to filter noise in the ECG obtained signals (21), and that is functionally linked to said amplifier; iv) at least one quartz crystal to generate electrical signals on a time basis (22), functionally connected to said microcontroller; v) at least one capacitor for the noise control signal functionally connected to said microcontroller (23); v) at least one capacitor for filtering noise signals functionally connected to said quartz crystal (24); vi) at least one capacitor to filter electrical noise signals functionally linked to the electrical supply module (25); vii) at least one group of resistances to control the current, functionally connected to the microcontroller, to the amplifiers and to previously mentioned electrical system power supply (27, 28, 29); and vii) at least one SPI flash memory which communicates with the microcontroller for storing said electrocardiographic events (30).

In another aspect of the present invention, the non-invasive portable device of the present invention is characterized in that the communications system (15) further comprises: i) a modem for a mobile communication system with integrated GPS data (17) for location data transmission of the patient carrying the portable noninvasive device of the present invention; and Bluetooth (18) module for wireless data transmission at short range.

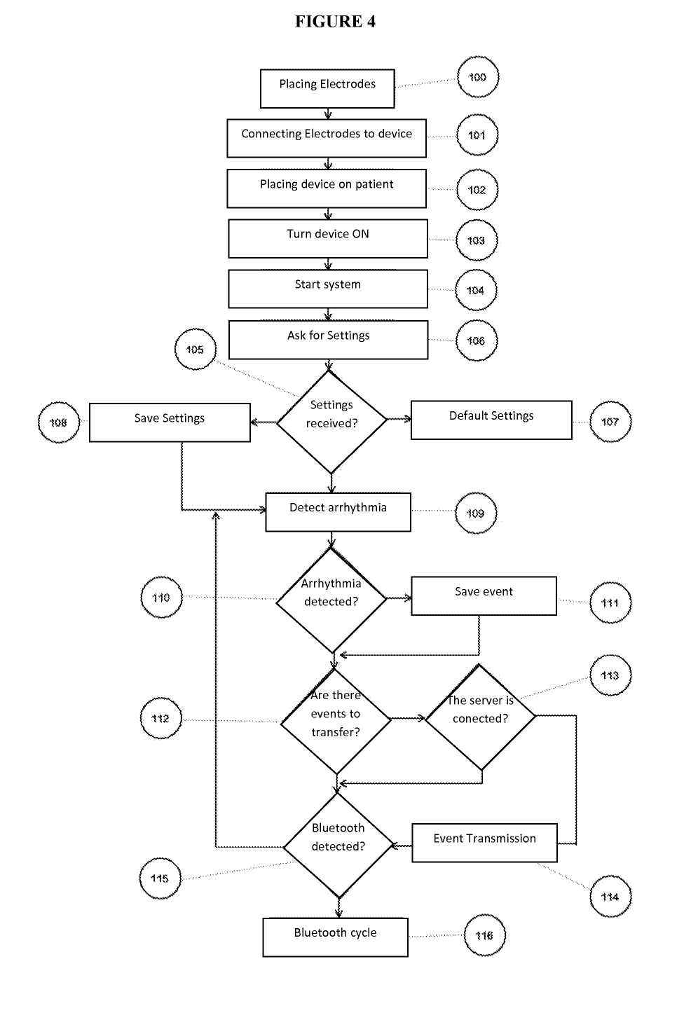

Finally, the present invention discloses a method for monitoring, storing, remote communication, diagnosis and alarm of electrographic events in a patient, which is non-invasive and directly transfers the data by means of a mobile data communication system (see FIG. 4), characterized in that it comprises the following steps:
i) (100) placing at least three electrodes on the patient's body (1) for the acquisition of the electrocardiographic signals;
ii) (101) connecting said electrodes to the portable non-invasive device, placed anywhere in the body (102) for acquiring, processing, storing, diagnosis and transmission of electrocardiographic signals of said patient (1);
iii) (103) turning said portable non-invasive device on, by activating the electrical feeding system (104);
iv) (106) entering a set of adequate configuration parameters for operating said portable non-invasive device, which comprise at least: the patient's maximum and minimum heart rate, the pre-recording and post-recording time of said electrocardiographic signals, and the device's ID (108), wherein if said parameters are not entered (105), the device runs on default settings (107);
v) Allowing for the patient (1) to carry on with his or her daily routine life while the portable non-invasive device (5) acquires (109), diagnosis (110), stores (111) and transmits said electrocardiographic signals by a mobile data communication system, Bluetooth and GPS location (112, 113, 114); and
vi) Allows the reception of said electrocardiographic signals by a medic or specialist, within a device which comprises mobile phones or computers, or directly connects to a Bluetooth device (115, 116).

### 5. DETAILED DESCRIPTION OF THE INVENTION

In connection with FIG. 1, it shows the system of the present invention in which a biomedical telemetry system, short for its English acronym, which allows for the monitoring of electrocardiographic events, remote identification and real time diagnosis of the patient's (1) cardiac functions, with the feature of being non-invasive and allowing a direct transmission of the gathered data by means of a wireless data communication system (6), which includes Bluetooth communication (3) and allowing GPS location (4).

The system of the present invention is characterized in that it comprises:
i) A portable non-invasive device (5) which may be placed anywhere in the body for acquiring, processing, storing and transmitting the patient's electrocardiographic signals;
ii) A wireless data communication network (6) which allows for the simultaneous data transmission by means of Bluetooth (3) and allows GPS location (4);
iii) A data storage software hosted on a server (8), characterized in that it can be accessed via the web (7); and
iv) An electrocardiographic signal visualization software characterized in that it allows real time visualization of the information gathered from the patient in at least one mobile device (9) or a computer (10).

The ability to integrate this system into a simple, lightweight, comfortable and safe device, with both physical and technological use by patients and medical specialists result in a valuable product of a high-knowledge, the result of investigations framed in a continuous line of technological development.

Continuing with FIG. 1, the device of the present invention initiates the information capture system based on the heart function obtained through conventional electrodes (2) which are fixed to the patient's body (1) placed at different established places, while remaining alert to the appearance of a cardiac event or heart disease.

Cardiac functions obtained by the information capture system are captured by the processing and analyzing system located in the non-invasive portable device (5), which analyzes the patient's cardiovascular behavior. The processing and analyzing system located in portable non-invasive device (5) consists of a signal acquisition system which when detecting an abnormality, immediately enables the recording equipment and, in real time, through a global system for mobile communications and mobile phones, through the wireless data communication network (6) that sends the information to the Specialized Center for Cardiovascular Monitoring (cardiologist) so that said specialist finds out what happened and can act immediately, either advising the patient (1), their families or even providing support and advice on clinical management. The wireless data communication network (6) of the present invention comprises a GPS tracking chip (4) for georeferencing the device (5) and the location of the patient (1).

The present invention has the ability to autonomously detect various types of cardiac abnormalities, including: locks, bradyarrhythmias, supraventricular tachycardia, atrial flutter, atrial fibrillation, supraventricular ventricular tachycardia, ventricular flutter and ventricular fibrillation, the latter being difficult to detect due to the QRS complex configuration which is the shape of the electrocardiographic signal lacking regular patterns.

Given the process of capturing the electrocardiographic signals is measured at skin level as extracellular potentials, the present invention sorts interfering factors that arise when working with bioelectric potentials and low voltage levels of the electrocardiographic signals. Interference is one of the factors that can alter the data obtained from the electrocardiographic signal, giving the possibility of erroneous or inadequate diagnoses. Therefore, to eliminate interference factors is one of the key parameters that requires a design that minimizes noise generated by electronic components, GPS and wireless communication systems. This is achieved by a specific provision of these elements, as detailed below, and its calibration and proper choosing using concepts defined from engineering and allowing the electrocardiographic signal to be processed and analyzed without difficulty.

In this regard, the present invention's hardware contemplates the features which allow the sorting of interference signals generated by the cellular networks. Given the above, the signal acquisition system located in the non-invasive portable device (5) consists of several elements for filtering and amplifying, as well as an energy rerouting system as a protection precaution against transient and defibrillation shocks. The hardware of the present invention includes Analog/Digital (A/D) converters, a microprocessor, USART and SPI serial communication devices, input and output ports and Flash drives. Furthermore, to detect different anomalies, the present invention has developed algorithms based on random behavior signal analysis tools in order to detect chaotic signals.

Referring to FIGS. 1 and 2, the non-invasive portable device (5) comprises: (i) a non-invasive cardiac event signal acquisition system; (ii) a system for digital storage and processing of such electrocardiographic signals which is functionally connected to the non-invasive electrocardiographic signal events acquiring system; (iii) a communications system between the digital processing signals diagnostic system and a wireless communication network for direct transmission of such electrocardiographic signals (15); (iv) a power system (16) for the device's energy supply (5); and (v) a global geo GPS (17) system.

In another aspect of the present invention, the portable non-invasive device (5) is characterized in that the non-invasive signal electrocardiographic events signal acquisition system comprises: at least three electrodes (2) for the electrocardiographic signals acquisition of which will connect directly to the patient's skin (1); an electrocardiographic signal amplifier functionally connected to said electrodes; a copper wire or other information conductor connection means material (11) between the electrodes and the electrocardiographic signals amplifier (12); at least one analog filter of said electrocardiographic signals (13) and a processing system equipped with a microcontroller (14).

Referring to FIG. 3, a portable non-invasive electronic device (5) is shown, which is characterized in that the digital signal processing module further comprises: a microcontroller (19) for signal processing comprising at least one 32-bit ARM architecture processor; at least one signal amplifier (20) functionally connected to said microcontroller (19); at least one capacitor (21) to filter out noise in the electrocardiographic signals obtained, functionally connected to said amplifier (20), wherein the capturing or sampling rate of said signals is at least 1 kHz; at least one quartz crystal (22) for generating electrical signals on a time basis, which in preferred embodiments may be 18 MHz, functionally connected to said microcontroller (19); at least one capacitor (23) for the signal noise control functionally connected to said microcontroller (19); at least one capacitor (24) for filtering noise signals, functionally linked to said quartz crystal (22); at least one capacitor (25) to filter electrical noise signals, functionally linked to the electric supply system (26); at least one group of resistances to control the current, functionally connected to the microcontroller (27), to the amplifiers (28) and to previously mentioned electrical system power supply (29); at least one flash drive (30) of SPI Bus communication (31) with the microcontroller for storing said electrocardiographic events.

Referring to FIG. 2, the portable noninvasive device (5), characterized in that the communication module further comprises: a modem for a mobile integrated data communication system for the transmission of data through a wireless network (15); and a GPS positioning system (17) which locates the patient's (1) position, wherein said patient is carrying the noninvasive portable device (5); a Bluetooth module for wireless transmission of data over short distances (18). The events detected by the digital signal processing module as well as the patient's position are transmitted using the modem for mobile communication. The Bluetooth module is used for reading the signal at close range.

In another aspect of the present invention, the connecting means (11) between the electrodes (2) and the aforementioned signals electrocardiographic amplifier (12) are characterized in that said means comprise: aluminum cables, copper cables, zinc cables or wires made from alloys between these metals which allow the transfer of said electrocardiographic signal.

Referring to FIG. 4, the steps of the method of the present invention for monitoring, storing, remotely communicating and providing alarms of electrocardiographic events in patients, which is noninvasive and provides direct data transfer via a system of mobile data communication, Bluetooth and GPS reception, are shown, which method is characterized in that it comprises the following steps:

i) (100) placing at least three electrodes (2) on a patient's body (1) for the acquisition of electrocardiographic signals;

ii) (101) connecting said electrodes to the non-invasive portable device (5), placed on any part of the body, for acquiring, processing, storing and transmitting ECG signals from said patient (102);

iii) (103) turning said portable non-invasive device on, by activating the before mentioned electrical feeding system and waiting for it to start its operation;

iv) (106) entering a set of adequate configuration parameters for operating said portable non-invasive device, which comprises at least: the patient's maximum and minimum allowable heart rates, the pre-recording and post-recording time of said electrocardiographic signals which indicate the amount of data stored after the event, and the device's ID (108);

v) allowing for the patient (1) to carry on with his or her daily life routine while the portable non-invasive device (5) acquires, diagnoses, stores and transfers said electrocardiographic signals by a mobile data communication system (15), Bluetooth (18) and GPS location (17); allows the reception of said electrocardiographic signals by a physician or specialist, through a device which comprises mobile phones (9), computers (10) or any personal computing device or tool.

In yet another aspect of the present invention, the portable acquisition, processing, storing, diagnosis, remote transmission and electrocardiographic alarm events system in patients, is able to detect at least 8 cardiac conditions selected from the group comprising: ventricular fibrillation, locks, brady arrhythmias, atrial supraventricular tachycardia, atrial flutter, atrial fibrillation, supraventricular ventricular tachycardia, ventricular flutter. This is achieved through the implementation of a software system that predicts changes in the frequency, rhythm variations, QRS duration and RR pauses identifying cardiac period. To detect heart disease, the non-invasive portable system (5) collects electrocardiographic activity through electrodes (2). It then analyzes the frequency of each heartbeat in the form of its recorded electrical signal. There are a number of parameters such as the minimum time between pulses to determine a case of tachycardia. The system continuously analyzes the measured variable against the programmed parameters. In the case of detecting that the variable exceeds the limit set by a parameter, such as heart rate is above 180 beats per second, the computer goes into discoverable mode. In said mode, it verifies that the anomalous variable was correctly measured; in which case the failure or abnormality event is generated. It is worth noting that these parameters are programmable on the device by the physician according to the needs of the patient who wears it.

Every type of heart disease has a specific presentation and recurrence which is indicated in the internal device parameters. These parameters involve an increase, decrease or absence of the heart rate, the non-appearance of an expected signal, the distortion of an electrocardiographic signal or the absence of a portion of the electrocardiographic signal.

The device is off when connected to the patient through the three electrodes placed at points in his or her chest at the location established by the skill of the art as appropriate area for placement, as understood by a physician or other persons of skill in the art. The device is then placed in a pouch, pocket or the like and activated. When the device is turned on, it first establishes a connection to the database platform where it requests a configuration profile. The server validates the encrypted information sent by the device (username and password). If the device is not in the database server, it will not be recorded and login to the database is not allowed. The device of the present invention operates with pre-event and post-event time parameters by default. If the device is registered in the database, a set of specific pre-event time and post-event time are sent back. Pre-event and post-event times are those indicating the amount of information recorded before and after a cardiac event. It is also possible to define specific parameters for each cardiac event based upon medical judgment. For example, a tachycardia is diagnosed when the heart rate (pulse) exceeds 220 beats per minute.

Then, the device enters an infinite loop where the cardiac signal generated by the patient is captured, filtered and amplified by the circuits. This signal is converted into a sequence of digital values which are continuously processed using algorithms that identify outliers. For example, the time difference between the two values of peaks of the QRS signal, corresponding to the heartbeat, can be analyzed as the difference in the number of digits between the two largest positive values present in the digital sequence. This value should be continuous and regular and should lie within specific ranges determined by a physician according to the patient wearing it; or show gradual changes, not sudden, in time. A sudden change is considered as such when the error exceeds a parameter defined by the physician and can be programmed according to the patient's condition. A sudden change, for example, is when a measurement sequence had 200 digits between the two largest positive values and the following measured sequence was only 100, which means that in the end there was a 50% change in the pulse.

If the heart rate exceeds 220 beats per minute, the device switches into event mode. The first thing it does is store the pre-event signal according to the specified time in its memory. It then continues to store the post-event signal for the indicated time in its memory. When the device already has the full signal stored in the memory, the latest GPS geo-referenced coordinate and the date and time when it happens is captured. With this data, it forms a data packet containing: time and date, data and GPS coordinates.

If there is no wireless connection at the time, the packet is stored in a non-volatile memory. When the connection is restored, the data is sent to the database located on a remote computing device. If there is a connection, the data is immediately sent to the database located on a remote computing device. Also, as appropriate, the attending physician may be contacted with an SMS message if it is as such programmed, or by some other means, about the occurrence of the event.

In the remote computing device, the signal and the registered coordinate is stored. As such, a specialist or a suitable person can immediately or at any time check the captured signal and decide the medical action to be taken for the patient.

It should be understood that the present invention is not limited to the described and illustrated embodiments, for as it will be apparent to one skilled in the art, variations and possible modifications which do not depart from the spirit of the invention, which is only found defined by the following claims.

1. A real time portable electrocardiographic diagnostic system in patients, characterized in that it comprises:
a. an electrocardiographic signals acquisition system;
b. a digital processing and storing system of electrocardiographic signals;
c. a wireless communication system that transfers information of the electrocardiographic signals to a communications network;
d. an electrocardiographic signals display system; and
e. a data processing software that is hosted on a server which can be remotely accessed.

2. The system of claim 1, wherein the electrocardiographic signals acquisition system comprises:
a. at least three electrodes for acquiring the electrocardiographic signals which attach to the skin of a patient;
b. an electrocardiographic signal amplifier functionally connected to said electrodes;
c. at least one analog filter for adapting said electrocardiographic signals;
d. a power module as an energy supply of the device; and
e. a connection means between the electrodes and the electrocardiographic signal amplifier.

3. The system of claim 1, wherein the storage and the digital electrocardiographic signal processing system further comprises:
a. At least one microcontroller for signal processing;
b. At least one signal amplifier functionally connected to said microcontroller;
c. At least one capacitor to filter the noise in the obtained electrocardiographic signals;
d. At least one quartz crystal to generate electrical signals on a timely basis, and which is functionally connected to said microcontroller;
e. A group of resistances to control the current, functionally linked to the microcontroller, amplifiers and power supply module; and
f. at least one flash drive for storing such electrocardiographic events.

4. The system of any of claim 1, wherein the communications module comprises:
a. A modem for transferring data via a cellular network;
b. At least one GPS system to send the patient location information of; and
c. A Bluetooth module for wireless data transmission over short distances.

5. The system of claim 2 wherein the connection means between the electrodes and the electrocardiographic signals amplifier are selected from the group comprising aluminum wires, copper wires, zinc cables or alloys produced between these metals which allow for the transfer of electrocardiographic signals.

6. The system of claim 1, wherein the system is capable of detecting at least 8 cardiac conditions selected from the group comprising: ventricular fibrillation, blocks, brady arrhythmias, atrial supraventricular tachycardia, atrial flutter, atrial fibrillation, supraventricular tachycardia and ventricular flutter.

7. The system of claim 1, wherein said system allows the storage of the electrocardiographic signal along with the positioning coordinate for the online analysis and response of an expert.

8. A method for diagnosing patients electrocardiographic events in real time, comprising the following steps:
a. placing at least three electrodes of a portable diagnostic system electrocardiographic events on any body part of a patient;
b. activating the portable electric power system module; and
c. setting the operation parameters of said portable non-invasive device.

9. The method of claim 8, wherein the portable system allows the delivery of electrocardiographic signals sent to an external device from the group comprising mobile telephones, computers, or any personal device or calculation tool.
